(19) United States
(12) Patent Application Publication
(10) Pub. No.: US 2024/0220815 A1
(43) Pub. Date: Jul. 4, 2024

(54) SYSTEM FOR THE AUTOMATED HARMONISATION OF STRUCTURED DATA FROM DIFFERENT CAPTURE DEVICES

(71) Applicant: AICURA MEDICAL GMBH, Berlin (DE)

(72) Inventors: Sebastian NIEHAUS, Buxtehude (DE); Daniel LICHTERFELD, Berlin (DE); Michael DIEBOLD, Berlin (DE); Janis REINELT, Leipzig (DE)

(21) Appl. No.: 18/043,418

(22) PCT Filed: Aug. 31, 2021
(86) PCT No.: PCT/EP2021/074031

§ 371 Date: Sep. 1, 2023

(30) Foreign Application Priority Data
Aug. 31, 2020 (DE) 10 2020 122 749.3

(51) Int. Cl. G06N 3/098

(52) U.S. Cl. CPC G06N 3/098

(57) ABSTRACT

A system for automated harmonization of structured data from acquisition devices, comprising
an input for input data sets in different, acquisition device-specific data structures,
a harmonization module embodying a harmonization model configured to transform a respective input data set from the respective system acquisition device-specific structure into at least one harmonized data set in a globally uniform, harmonized data structure of the system,
a preprocessing module embodying a preprocessing model configured to transform data from a harmonized data set into data in a model-specific data structure, in particular to perform feature reduction so that a data set with preprocessed data in the model-specific data structure represents fewer features than a corresponding data set in the globally uniform structure, and
an automated processing facility configured to automatically process preprocessed data in the model-specific data structure to classify and to generate a loss measure representing a possible processing inaccuracy (loss) and to output it optionally to the harmonization model or the preprocessing model.

The invention relates to a system for automated harmonization of structured data from various acquisition devices.

Acquisition devices can be, for example, imaging devices in medical technology such as tomographs or the like, but also measuring devices, analyzers, and other devices that provide data typically structured in relational data sets. One problem for technical data processing is that even data from similar devices for the same purpose, e.g. data from tomographs—despite some de facto standards such as FIHR (Fast Healthcare Interoperability Resources)—do not necessarily have the same structure or format. This makes it difficult to have a uniform technically automated evaluation or analysis of these data—especially an automated analysis.

To solve this problem, a system for automated harmonization of structured data from different acquisition devices is proposed, which includes the following components:
an input for input data sets in different, acquisition device-specific data structures, i.e. each in a structure as supplied by a respective acquisition device,
a harmonization module embodying a harmonization model which is machine-generated and configured to convert a respective input data set from the respective acquisition device-specific structure into at least one harmonized data set in a globally uniform, harmonized data structure of the system,
a preprocessing module embodying a preprocessing model which is machine-generated and configured to convert data from a harmonized data set in the globally uniform harmonized data structure into data in a model-specific data structure, in particular to perform feature reduction so that a data set with preprocessed data in the model-specific data structure represents fewer features than a corresponding data set in the globally uniform structure, and
an automated processing facility configured to automatically process preprocessed data in the model-specific data structure, in particular to classify and to generate a loss measure representing a possible processing inaccuracy (loss) and optionally to output it to the harmonization model or the preprocessing model.

The system according to the invention serves to enable its automated processing facility to process data from different kinds of input data sets, which may originate from different sources, equally by means of one or more classification models or one or more regression models. The automated processing facility thus embodies one or more classification models or regression models, each of which is preferably in the form of a neural network.

Acquisition devices can be devices, such as tomographs, but in particular also data processing devices that merge data from different sources into a relational data set. The merged data can be medical history data, patient master data, laboratory values from different laboratories, image or model data from different modalities such as tomographs, etc.

Accordingly, the formats of the various data may differ, although they may basically concern the same parameter, such as a leukocyte count. But the structure of the relational data sets can also be different, depending on how the various partial data sets from the different sources have been combined to form a respective relational data set.

For these reasons, the input data sets can be very different, even though they may concern basically the same data.

For automated processing, the problem arises that data sets that differ in structure and in the form of representation of underlying values, such as laboratory data, etc., cannot be assigned to specific classes with a high probability of membership, i.e., they cannot be reliably classified.

Data supplied by an acquisition device each form an input data set, which typically comprises several partial data sets and has a structure that deviates from a globally uniform, harmonized data structure specified for the system.

An acquisition device may be a device that generates data, such as image data representing a captured image. An acquisition device may also be a data processing device that is used to combine data from various sources into a data set (which may serve as an input data set for the system according to the invention).

The data in the partial data sets can represent, for example, acquired images or volume models, as well as patient data such as age, gender, height, weight, blood group, BMI, medical history, etc., or laboratory data, e.g. as the result of a blood test.

Subject natter of the invention therefore is a system for automated harmonization of data sets originating from different acquisition devices. In particular, relational data sets comprising data from different sources, e.g. from imaging devices, in the form of partial data sets are concerned.

Incoming data, for example from a data acquisition system, is first converted by a harmonization module into a globally uniform, harmonized data structure. A preprocessing module then converts the uniformly structured data into data with a model-specific data structure. This data in the model-specific data structure is finally fed to an automated processing facility, e.g. a classifier or regressor, which can be implemented in the form of a parametric model (neural networks, logical regression, etc.) or a non-parametric model (decision tree, support vector machines, gradient boosting trees, etc.).

The automated processing facility implements a classification or a regression model. Model changes to the classification model or a regression model implemented by the automated processing facility are implemented in a manner known per se using prediction error, preferably as a supervised learning algorithm. For example, the prediction error can be determined in a known manner with a loss function and the change of the classification model or a regression model implemented by the automated processing facility can be done in the case of a neural network by adjusting the weights in nodes of the layers by backpropagation.

The prediction error of the automated processing facility shall be as small as possible. The prediction error of the automated processing facility is not only based on the processing of the data provided by the preprocessing module by the automated processing facility itself, but also on the processing of the input data sets by the harmonization module and the processing of the harmonized data sets by the preprocessing module. Therefore, the prediction error is used not only for adjusting the classification or regression model implemented by the automated processing facility, but also for optimizing the harmonization model embodied by the harmonization module and the preprocessing model embodied by the preprocessing module. Both the harmonization module and the preprocessing module are thus capable of learning, i.e. can be trained by machine learning.

Thus, the training of the harmonization module and the preprocessing module are performed taking into account the prediction error of the automated processing facility.

Preferably, the harmonization module embodies a trained neural network, in particular a multilayer fully interconnected perceptron or a deep Q-network.

Preferably, the preprocessing module embodies a trained neural network, in particular an autoencoder.

Preferably, the harmonization module is connected to a plurality of pre-processing modules and each of the pre-processing modules is connected to an automated processing facility.

Preferably, the or each automated processing facility for providing feedback to the harmonization module is connected thereto.

Preferably, the or each automated processing facility is connected to the respective upstream preprocessing module for providing feedback thereto.

In accordance with the invention, an interconnection of multiple systems of the type described herein is also proposed, in which the systems are interconnected to exchange parameter datasets to enable federated or collaborative machine learning. The parameter datasets include parameter values representing training-generated weights of the harmonization or preprocessing models embodied by the harmonization or preprocessing modules.

### The Harmonization Module

The harmonization model embodied by the harmonization module is a model for combining and assigning the data represented in the partial data records to partial data records of a uniform, harmonized data structure that facilitates reliable processing of the data by the automated processing facility. The assignment decision—i.e. the decision which data from the partial data records of the respective input data record are assigned to the partial data records of a data record in the globally uniform, harmonized structure—is thereby modeled as a classification. The harmonization module therefore preferably embodies a classifier. This classifier can be constructed as a 3-layer perceptron with 12 nodes per layer, which are fully connected to each other. The activation function of the nodes is preferably nonlinear, e.g. a leaky ReLU function. The data basis for the assignment decision is data acquired in context and the origin of the respective input data set. However, the harmonization model is preferably not fully approximated, but is represented as a rule-based structure that is extended by an approximated (trained) model.

In the trained state of the harmonization model embodied by the harmonization module, the harmonization module is configured to search for the most suitable partial data record of the globally uniform, harmonized data structure of the system for a suitable assignment of partial data records from an input data record to a partial data record of the globally uniform, harmonized data structure of the system. The search is preferably implemented as a hierarchical search, whereby the search behavior is determined by a deterministic heuristic derived from a metaheuristic or by an agent with a search behavior that was approximated via reinforcement learning.

The search behavior is preferably deterministically constrained by a reward function composed of the feedback from the automated processing facility and a defined rule set. The feedback from the automated processing facility can be, for example, the loss determined by means of the loss function, which results as a consequence of the prediction error as it occurs in the context of the supervised learning of the automated processing facility.

The search space within which the harmonization module searches for a suitable assignment is defined by the hierarchical structure of the specified globally uniform, harmonized data structure of the system, which is the goal of harmonization. The given globally uniform, harmonized data structure of the system represents the environment for the preferred reinforcement learning. In the case of reinforcement learning, the training of the harmonization module can be limited by predefined action spaces and thus optimized.

The predefined action spaces for reinforcement learning can represent a defined rule set. This can also be realized as a dictionary for the assignment of the partial data records of a respective input data record to partial data records of the specified globally uniform, harmonized data structure.

The automated processing facility providing the feedback for training the harmonization module (i.e., e.g., the prediction error or loss) may be a black-box function that returns only an evaluation of the input parameters and a deviation for the target value.

In a training phase, both the harmonization model embodied by the harmonization module and the preprocessing model embodied by the preprocessing module are optimized by means of the feedback from the automated processing facility—but not simultaneously, but one after the other—i.e. only one module at a time. Feedback from the automated processing facility, e.g. the classifying neural network, is used for this purpose, in particular the loss, which should be as low as possible.

The first module that processes the incoming data is the harmonization module. This can, for example, embody a metaheuristic that forms a (decision) tree structure. During training, points (weights) are formed for each node connection (connection between two nodes in the decision tree) of the metaheuristic depending on the feedback (especially the loss) provided by the classifying neural network. The strongest node connections, i.e., those with the highest weights or the most points, are eventually retained and form a deterministic heuristic after training. Adjusting the node connections is done until a suitable deterministic heuristic is formed.

Thus, the metaheuristic can be an original decision tree with all possible node connections present. Training results in a deterministic heuristic, which can be a decision tree that has only unique edges.

Such a deterministic heuristic can also be generated manually, but this would be very time consuming. According to the invention, a metaheuristic is used instead, which enables a heuristic search.

If the harmonization model is a metaheuristic that forms a tree structure that is formed during training (see above: points are given for the respective node connections in order to let less relevant node connections “die off” in this way), the optimization is initially performed stochastically by randomly mapping features from the system-specific structure to features in the globally uniform structure and then finally considering the resulting classification result and forming and optimizing the structure at least initially according to a kind of trial-and-error procedure.

Harmonization models generated in this way, e.g., deterministic heuristics with a tree structure generated from a metaheuristic by means of training, can be collected and aggregated for various systems that are not, incidentally, locally interconnected and made available to other systems in each case, so that a locally generated harmonization model can be compared with one (or with several) locally stored harmonization models with respect to the classification success by the automated processing.

During the training of the harmonization model, possible mappings based on the hierarchical structures of the coding system are explored and the result changes of downstream processing models (e.g. machine learning models) are used as feedback for the harmonization model.

Different harmonization models of different harmonization modules can be approximated in a decentralized manner over multiple instances using federated or collaborative learning by exchanging parameter datasets between the harmonization modules that contain the parameter values resulting from the training, in particular the weights of the nodes of a respective neural network.

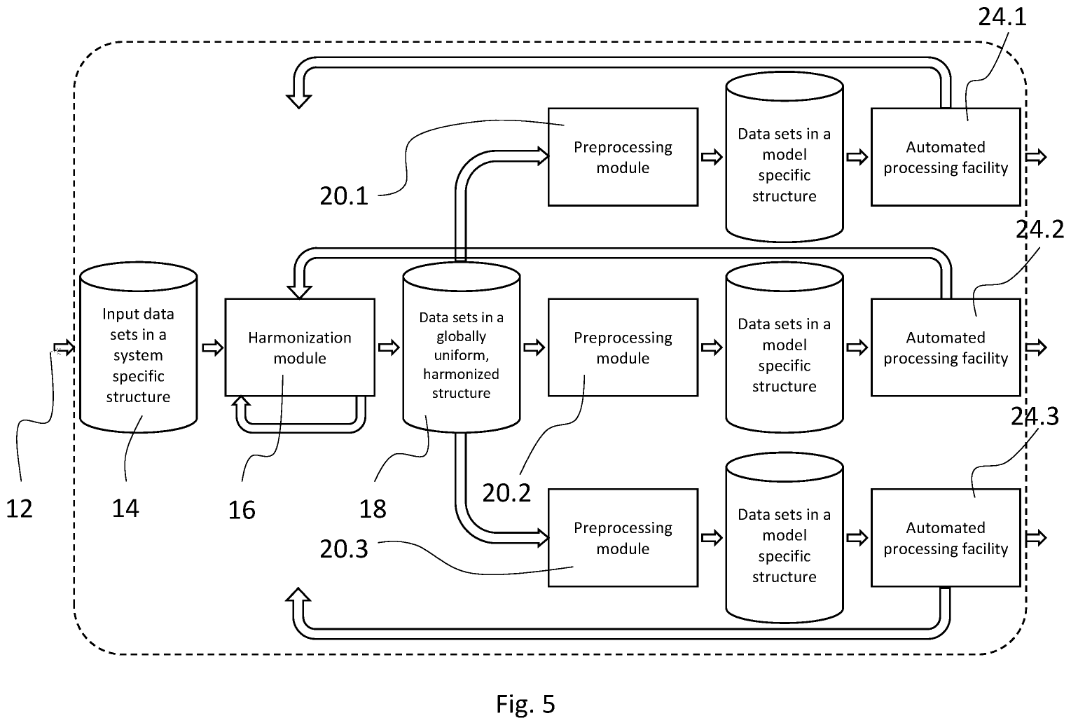

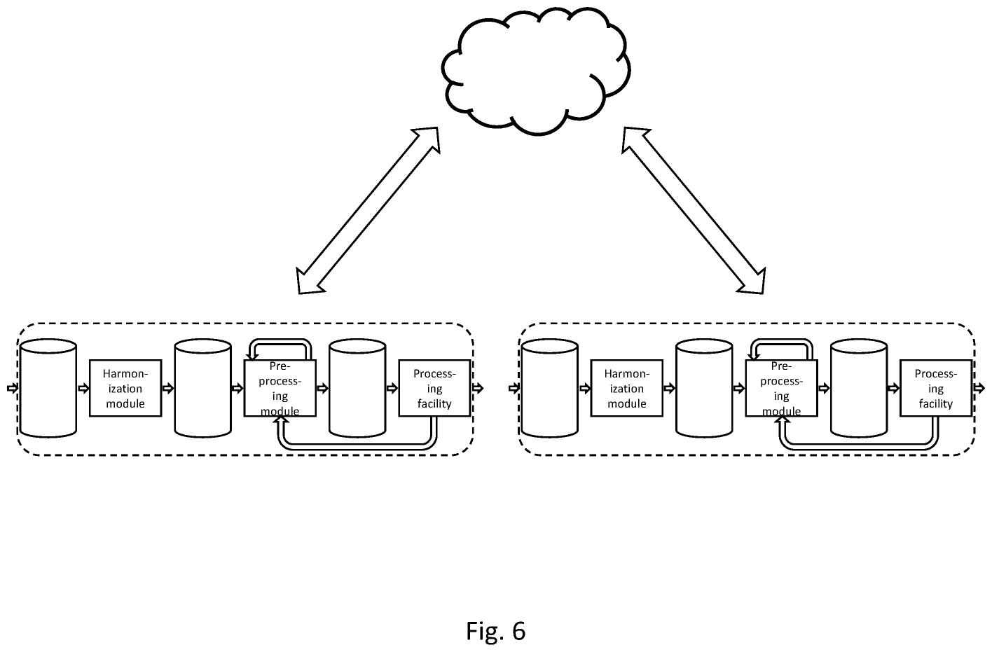

Data communication for exchanging such parameter data sets between the individual harmonization modules can take place via a global server (see FIG. 5 or 6) or directly from module to module.

A prerequisite for such federated or collaborative training of different harmonization or even preprocessing modules is that the respective modules embody models with the same topology or structure.

Alternatively, the harmonization model can be generated by reinforcement learning, which is based on a Markov model with states, state transitions and a virtual agent that induces state transitions. For this reinforcement learning the environment is fixed. The environment is, on the one hand, the input data sets with their partial data sets specified during training and, on the other hand, the specified globally uniform data structure to which the partial data sets and the data contained therein are to be mapped. As a result, the trained harmonization module embodies mapping rules for mapping the incoming data in their respective system-specific data structure to the globally uniform data structure. The mapping rules can be defined by a heuristic search or a neural network trained by reinforcement learning.

The harmonization module can be the same for several classification models each and can therefore be optimized with feedback from several classification models (maximum likelihood method).

The harmonization model is preferably implemented in the form of a deep Q-network. This has the topology of a multilayer perceptron with an input layer and an output layer and two hidden layers in between. The perceptron is trained by reinforcement learning, especially Q-learning, and is thus a deep Q-network. Training by means of Q-learning implies agents that can cause state transitions, e.g. the assignment of a partial data set of the input data set to a partial data set of the harmonized data set. The training is based on the fact that, as a result, favorable (advantageous) state transitions are rewarded with a reward for the agent. In the context of Q-learning, an action space can be given to a respective agent, so that the agent does not receive a reward for state transitions outside the action space. The action spaces specified in the context of Q-learning represent a rule base that underlies the harmonization model and thus the harmonization module.

Preferably, such a rule base is given, as this speeds up training and helps to avoid misassignments.

The reward also depends on the feedback that is returned to the harmonization model by the automated processing facility according to the invention. This feedback depends on the prediction error (in particular the loss) that results from training the automated processing facility on the basis of training data sets (ground truth). The prediction error of an automated processing facility designed as a classifier or regressor during training does not directly depend on the training data sets used as input data sets, since these input data sets, before being fed to the automated processing facility, are first processed by the harmonization module and by the preprocessing module. The respective prediction error, on which the feedback to the harmonization module and the pre-processing module is also based, thus depends on the processing of the input data sets in the harmonization module, in the preprocessing module and in the automated processing facility.

The training of the harmonization module or the preprocessing module is performed simultaneously with the training of the automated processing facility based on input data sets that form a ground truth. By comparing the classification result or the regression result provided by the automated processing facility with the ground truth data, the corresponding prediction error or loss can be determined.

During training, however, the feedback from the automated processing facility is not fed to both the harmonization module and the preprocessing module at the same time, but only to one of the two modules at a time, so that either the harmonization module or the preprocessing module is trained together with the automated processing facility.

The globally uniform, harmonized structure of the data sets that the harmonization module provides as output is predefined and can be FHIR-compliant, for example.

### The Preprocessing Module

The preprocessing module is preferably configured to perform feature reduction by way of principle component analysis (PCA). This can be done, for example, by having the preprocessing module embody an autoencoder that maps larger feature vectors to smaller feature vectors. The input layer of the autoencoder would then have as many nodes as the input vector has dimensions, and the output layer of the autoencoder would have a correspondingly smaller number of output nodes.

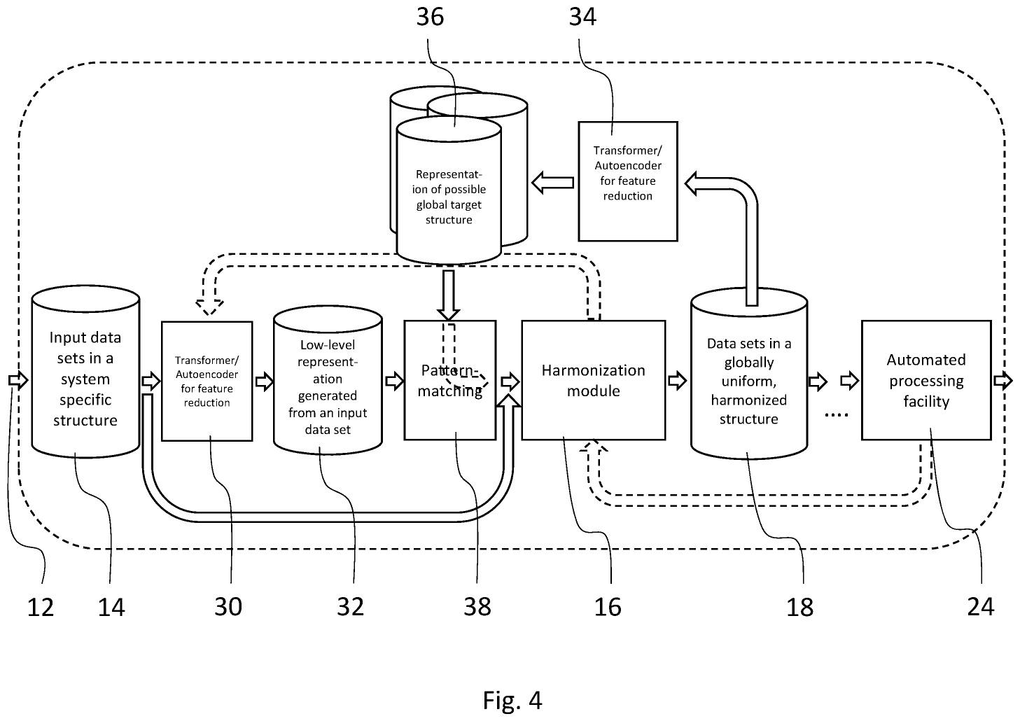

The preprocessing model, e.g. the autoencoder, is also trained with the aid of feedback from the automated processing facility, e.g. a classifier that embodies a classification model in the form of a classifying neural network, in order to arrive at preprocessed data records in a model-specific data structure that lead to the best possible classification by the automated processing facility in each case. The preprocessing model embodied by a respective preprocessing module is specific to a respective classification model of the automated processing facility, as can be seen, for example, in FIG. 4.

Preferably, the preprocessing module is configured to transfer data from a partial data set of a harmonized data set into a partial data set in which the data is present in a feature-reduced form.

Also, for training the harmonization module, the automated processing facility providing the feedback (i.e., the prediction error or loss, for example) can be a black-box function that returns only an evaluation of the input parameters and a deviation for the target value.

In a preferred embodiment, the system additionally comprises a module, in particular a transformer module, for generating a low-level representation of a respective input data set. The low-level representation of a respective input data set represents the structure of the input data set abstracted from the values contained in the input data set, in which the values are embedded. Low-level representation of a respective input data set can be fed to the harmonization module in addition to the input data set itself to improve the transformation of the input data set into a data set in the globally uniform structure.

In this regard, it is advantageous if the system additionally comprises a second module, in particular a transformer module, for generating multiple low-level representations of a harmonized data set, as well as a pattern matching module configured to determine which of the feature-reduced abstracted representations of the global target structure in question best matches the low-level representation of the input data set.

A transformer module may be implemented as a neural network in the form of a transformer model. Transformer models are known to the skilled person and have an encoder-decoder structure with an encoder part and a decoder part. The encoder part generates increasingly abstract feature vectors from an input data set, which are transformed again by the encoder part into output data sets that are concrete representations. In a transformer, self-attention layers are assigned to each of the layers (hidden-layer) of the encoder part; see http://jalammar.github.io/illustrated-transformer/.

A transformer module implementing a transformer model for generating multiple low-level representations of a harmonized data set has the property that its encoder part generates multiple low-level representations of the input data set of the transformer due to the self-attention layers. According to a preferred embodiment, this property is used to perform pattern matching between a low-level representation of the system's input data set with different low-level representations of a data set in the globally unified structure generated by the second transformer from the data set in the globally unified structure as the second transformer's input data set.

In this way, the best matching positions for values contained in the input data set of the system (i.e., the input data set in the capture device-specific structure) can be found in the data set in the harmonized structure.

The invention will now be explained in more detail by means of examples of embodiments with reference to the figures. From the figures shows:

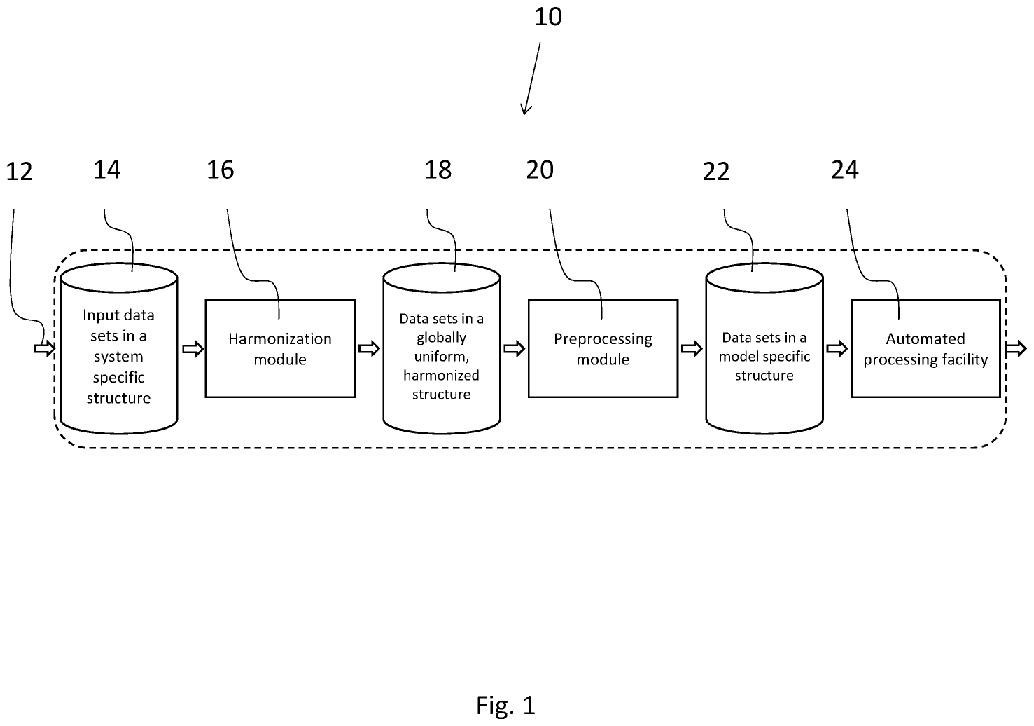

FIG. 1: A schematic overview of the system according to the invention;

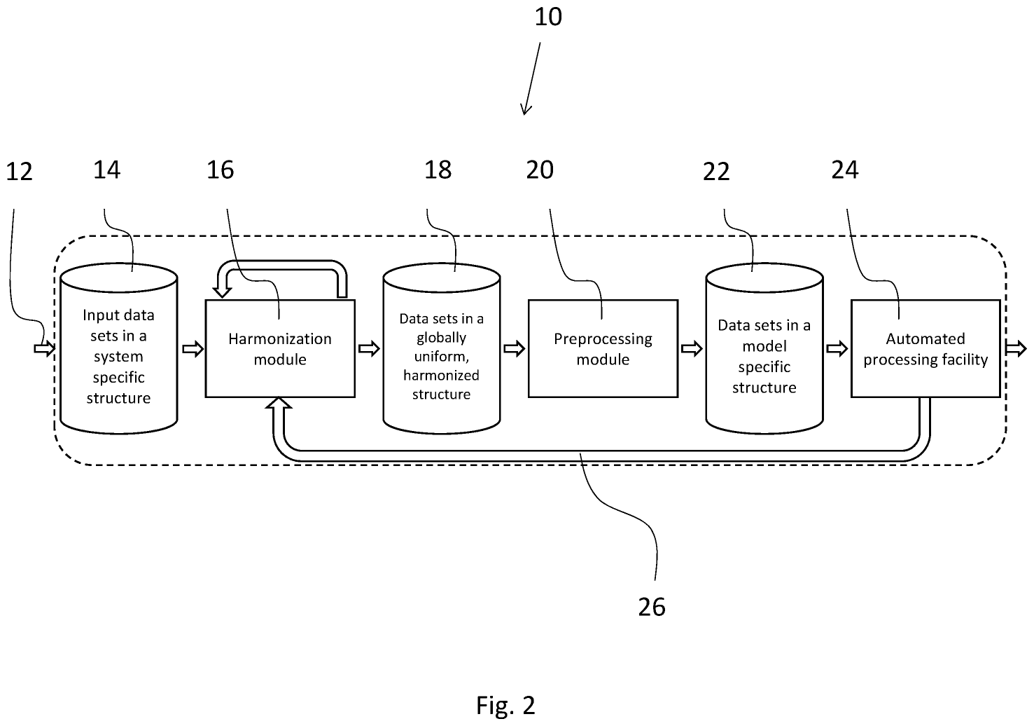

FIG. 2: A sketch explaining the training of the harmonization module;

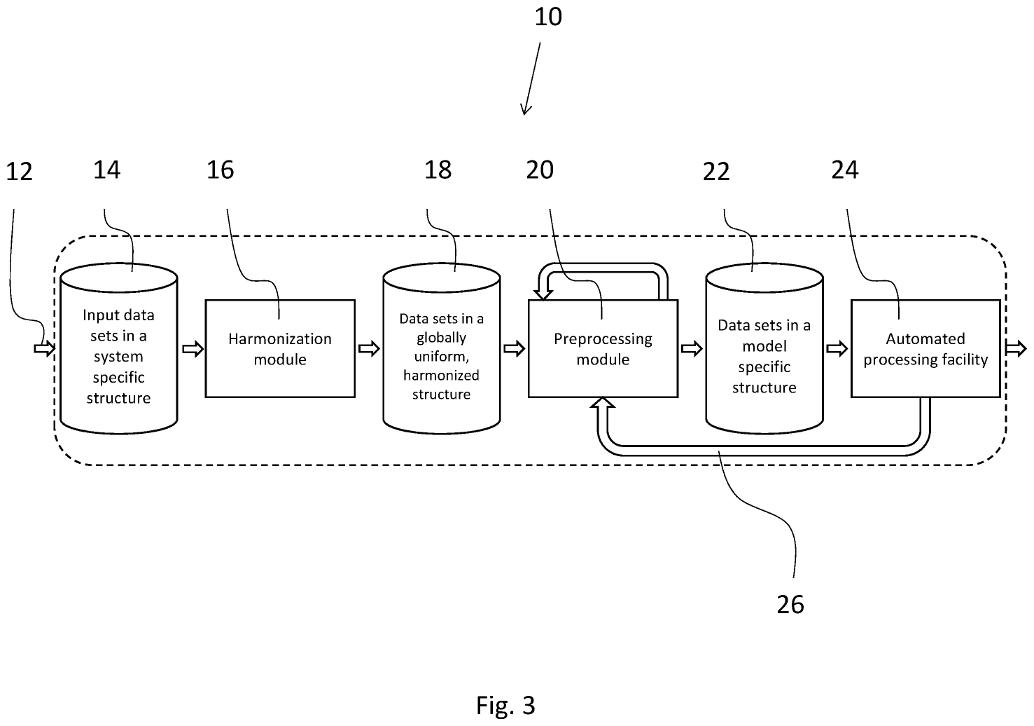

FIG. 3: A sketch explaining the training of the pre-processing module;

FIG. 4: A schematic overview of an extended system according to the invention;

FIG. 5: A sketch illustrating the training of the harmonization module using feedback from various automated processing facilities;

FIG. 6: A sketch illustrating how trained preprocessing models can be optimized by different preprocessing modules in a federated learning fashion; and

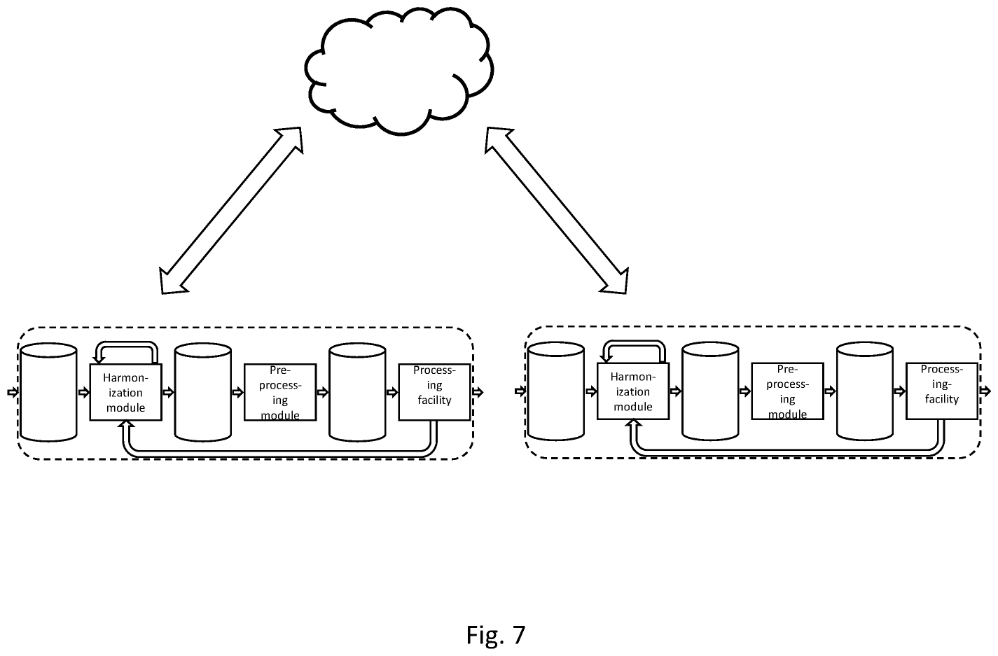

FIG. 7: A sketch illustrating how trained harmonization models can be optimized by different harmonization modules in a federated learning fashion.

FIG. 1 shows a system 10 for automated harmonization of structured data from different acquisition devices.

The system has an input 12 for an input data set 14 in an acquisition device-specific structure, i.e., in a structure as provided by a respective acquisition device.

The system further comprises a harmonization module 16 embodying a harmonization model that is machine-generated and configured to transform the data from the respective acquisition device-specific structure into at least one harmonized data set 18 a globally uniform data structure of the system. The structure of a data set is referred to herein simply as a structure or data structure. A harmonized data set 18 in a globally uniform structure of the system thus has a harmonized data structure.

The system further comprises a preprocessing module 20 embodying a preprocessing model that is machine-generated and configured to transform data from a harmonized data set 18 in the globally uniform harmonized structure into preprocessed data 22 in a model-specific data structure, in particular to perform feature reduction such that preprocessed data 22 in a preprocessed data set in the model-specific data structure comprises fewer entries than a corresponding data set in the globally uniform harmonized structure.

Further, the system comprises an automated processing facility 24 configured to automatically process preprocessed data 22 in the model-specific data structure, in particular to classify and generate a loss measure representing a possible processing inaccuracy (loss) or a possible prediction error (prediction error) and to output it as feedback 26 selectively to the harmonization module 16 or the preprocessing module 20. For example, the automated processing facility 24 provides as an output a membership or a probability of membership of the input data set to a class—for example, a disease—for which the automated processing facility has been trained.

For example, the automated processing facility 24 is configured to determine an affiliation probability value representing a respective affiliation probability determined for, for example, a class. These membership probability values represent a prediction that may be compared to training data providing a ground truth from corresponding input data sets for the system 10 during supervised learning to determine a prediction error and/or a loss. The prediction error or loss may be communicated back to the harmonization module 18 or the preprocessing module 20 by the automated processing facility 24 as feedback. This allows both the harmonization module 18 and the preprocessing module 20 to automatically optimize the system 10 during training such that the membership probability determined by the automated processing facility 24 for respective one class is as large as possible and the prediction error and/or loss is as small as possible.

An input dataset 14 in an acquisition device-specific structure is a heterogeneous relational dataset composed of multiple heterogeneous partial datasets and may be in an XML format, for example. For example, an input data set may include an image data set as a partial data set representing an image or solid model represented by pixels or voxels. Another partial dataset of this input dataset may contain metadata about the image dataset, for example, data representing acquisition time, acquisition medium (modality), acquisition parameters such as step size or energy, etc. Another partial data set may represent, for example, laboratory results of a blood test or an ECG of the same patient to which the other partial data sets also belong.

For example, the input data set 14 can contain anamnesis data (admission diagnosis, previous illnesses, age, place of residence, BMI, allergies, etc.) and various laboratory values (number of leukocytes, various antibody concentrations, etc.) for each patient.

### The Harmonization Module 16

The input data sets 14 from different sources—e.g., from different clinics—can be structured very differently and also contain different types of partial data sets.

The function of the harmonization module 16 is to convert different input data sets 14 into at least one harmonized data set 18 in a uniform, harmonized data format, thereby generating a harmonized data set 18 for each input data set 14.

For this purpose, the harmonization module 16 may embody, for example, a deterministic heuristic that assigns data from the partial data sets of the input data set to corresponding partial data sets of a harmonized data set in the manner of an assignment tree. The deterministic heuristic is generated from a metaheuristic representing a general tree structure in which many nodes of an assignment tree are connected to many other nodes via many node links. In supervised learning, the number of node connections is then reduced to effect a deterministic mapping of partial records of an input dataset to partial records of a harmonized dataset.

The deterministic heuristic can also be approximated by a neural network—i.e. implemented in the form of a neural network. A suitable network is, for example, a fully networked perceptron that is trained using reinforcement learning. A deep Q-network trained using Q-learning is particularly suitable. Q-learning is a form of reinforcement learning in which the Q-learning algorithm can be given underlying agents action spaces. These action spaces define a given rule base and structure a decision tree given by metaheuristics. The Q-learning algorithm is based on virtual agents that induce state transitions (corresponding to the transitions in the decision tree) and receive a higher reward if the induced state transitions lead to a better result—for example, a smaller prediction error of the automated processing facility. The predefined action space allows certain state transitions to be penalized. In addition, Q-learning can be more efficient because the number of possible states becomes smaller—i.e., the decision tree as an untrained metaheuristic allows for fewer possible decisions.

For example, a 34-layer perceptron with 12 nodes per layer is suitable for the implementation of a deep Q-network. Such a perceptron has an input layer, an output layer and two hidden layers in between. The 12 nodes of each layer are fully connected to the nodes of the adjacent layer(s). The activation function of the nodes is preferably nonlinear, for example a ReLU function and in particular a leaky ReLU function.

Alternatively, the harmonization module 16 may embody a Bayes net, in particular a Markov model and especially a hidden Markov model, generated by supervised learning. Also, the Bayes net or the Markov model may be approximated by a perceptron—that is, implemented in the form of a perceptron and trained by supervised learning.

To train the deterministic heuristic or the Markov model, the prediction errors occurring during the training of the automated processing facility, for example in the form of a loss determined by means of a loss function, are transmitted back to the harmonization module and the deterministic heuristic or the Markov model or the perceptron representing these are trained by means of reinforcement learning in such a way that the harmonized data sets generated by the harmonization module lead to the smallest possible prediction error or loss for a respective class. The prerequisite for this is that the training is carried out with fundamentally suitable input data sets for which it is known (as ground truth) to which class the data contained in the respective input data set are to be assigned.

If a different method for determining the leukocyte count is used in a clinic A and in a clinic F respectively than in the other clinic, which does not provide comparable values, both the type of representation (coding) of the leukocyte counts and the data structure containing the representative data may be different. Accordingly, input datasets originating from different clinics may differ both in terms of the form of the data and the position in which the data are stored in the dataset. In order to be able to process the input data sets with an automated processing facility, e.g., a classifier or regressor formed by a neural network, the different input data sets must be converted into a globally uniform, harmonized data structure that is predetermined for the system.

For example, the goal of classification or regression using the automated processing facility 24 may be to determine the risk of infection with hospital germs and/or the expected length of stay and/or to determine a score for the expected risk of hospital germs based on data from a respective input data set.

To make this possible in the result, each input data set 14 is first fed to the harmonization module 16. This embodies a trained harmonization model; see FIG. 1.

The harmonization model is trained by the feedback from the automated processing facility 24 such that the harmonization module 16 recognizes partial data sets of an input data set and transforms them into an appropriate partial data set of the globally uniform, harmonized data structure of the system; see FIG. 2.

With respect to the data representing values (e.g., pixels, voxels, laboratory values, etc.) within a respective partial data set, the harmonization model is trained with the aid of feedback from the automated processing facility such that the harmonization module recognizes the similarity between the values represented by the data and thus converts the data into a uniform form of representation (code system). For the leukocyte count, for example, the harmonization model is trained to divide the data representing values into two forms of representation (code systems)—that is, into two different subsets of the globally uniform, harmonized data structure of the system. The reason for this is that equal treatment of the values represented in different ways—even if they each represent leukocyte counts—will result in a worse classification with a lower probability of membership. Equivalent treatment of the values from the different measurement methods results in a worse membership probability value (worse reward, greater loss) because the classifier cannot map differently represented values to individual classes as accurately. The assignment to different partial data sets leads to the fact that the partial data sets are also classified differently, i.e. are fed to a different classification model in each case. Alternating classification models ensure that there is no over-adaptation in favor of one classification model. The exchange between clinics allows already trained parameters to be used and thus a transfer effect to be exploited.

The preprocessing module 20 The preprocessing model 20 provides for a selection of the relevant parameters and translates both types of leukocyte values into a uniform format. In particular, the relevant parameters are model-specific.

The harmonized data sets 18 are fed to the preprocessing module 20; see FIG. 1. The preprocessing module 20 is designed to convert at least some partial data sets of a respective harmonized data set 18 into preprocessed data 22 in a model-specific data structure, in particular to perform a feature reduction which is model-specific in that it is adapted to a (multiclass) classification model represented by the automated processing facility 24, because the preprocessing model has been trained (only) with the feedback of the respective downstream automated processing facility 24.

For example, the preprocessing module 20 is configured to perform feature reduction for those partial data sets that include image data representing pixels or volume data representing voxels. Such partial data sets may represent, for example, a plurality of features caused by noise, which may be eliminated by way of feature reduction so that a preprocessed partial data set of the preprocessed model-specific data set represents, for example, a less noisy image.

To this end, the preprocessing module 20 may be configured to perform principal component analysis, for which the preprocessing module may be an autoencoder. Possible implementations are described, for example, in Kramer, M. A.: “Nonlinear principal component analysis using autoassociative neural networks.” AIChE Journal 37 (1991), no. 2, pp. 233-243 or Matthias Scholz “Nonlinear principal component analysis based on neural networks,” Diploma thesis, Humboldt University of Berlin, 2002.

The purpose of the model-specific processing of a respective unified harmonized data set 18 by the preprocessing module 20 is to prepare data from particular partial data sets of the harmonized data structure for subsequent processing by the automated processing facility. If the preprocessing module embodies an autoencoder, the autoencoder may be trained to scale laboratory data from a respective partial data set of the harmonized data set to a uniform scale. It is also possible that the autoencoder is additionally or alternatively trained to reproduce only individual laboratory data on the output layer and thus, as a result, to filter the laboratory data given to the input layer of the autoencoder such that only laboratory data more relevant for subsequent processing by the automated processing facility are passed to it. If the partial data set supplied to the pre-processing module contains image data, the autoencoder embodied by the pre-processing module may also be trained to suppress noise represented in the image data or to enhance contrasts in the image data, thereby rendering a matrix-like representation of the respective image on the output layer that results in more reliable processing by the subsequent automated processing facility.

The pre-processing module 20 is also initially trained by feedback from the respective downstream automated processing facility 24, but not simultaneously with the harmonization module 16; see FIG. 3.

Also, the training of the pre-processing module 20 embodying an autoencoder is based on feedback from the automated processing facility such that the prediction error of the automated processing facility is as small as possible with respect to the ground truth (given by the input data sets during the training of the system 10 comprising harmonization module 16, pre-processing module 20 and automated processing facility 24). As explained above, a loss determined by means of the loss function known per se may be used as a measure of the prediction error and used as feedback for training the harmonization module 16 or the preprocessing module 20.

While the harmonization module 16 embodies, for example, a perceptron that is trained by way of Q-learning and thus represents a deep Q-network in the result, the preprocessing module 20 embodies, for example, an autoencoder that is trained by way of backpropagation. In this regard, both the training of the harmonization module 16 and the training of the preprocessing module 20 are also based on the prediction error provided by the automated processing facility 24 (as a classifier or regressor) over the input data sets used in training the system, which represents a ground truth.

The input data sets with different structure contain data (values) embedded in different structures. This means that values for the same parameters can differ not only by their data format, but can also be located at different positions in the respective input data set. In order to transfer the input data sets into a globally uniform structure, the values must be transferred from the respective position in the input data set to the corresponding position in the data set in the globally uniform, harmonized structure.

To facilitate this, an extended system 10′ is provided for automated harmonization of structured data from different acquisition devices, as exemplarily illustrated in FIG. 4. In addition to the same components as the system 10 described in FIGS. 1 to 3, the extended system 10′ has additional components which serve to reduce a respective input data set to its structural features by converting the respective input data set into a low-level representation and comparing and evaluating the low-level representations of the data sets in a globally uniform, harmonized structure by pattern matching.

To generate a low-level representation of a respective input data set, a first transformer module 30 representing a transformer model is provided. A transformer model is a form of a neural network with an encoder-decoder structure. The first hidden layers (hidden layers) of the transformer model following the input layer form an encoder and generate increasingly abstract feature vectors from the input data, which are then typically processed back into more concrete output data sets in a decoder portion of the transformer model. In a Transformer, Self-Attention layers are assigned to each of the layers (hidden-layer) of the encoder part; see http://jalammar.github.io/illustrated-transformer/.

The feature vectors generated by the encoder part of the transformer model represent feature-reduced low-level representation 32 of the input data set, which is used for the extended system 10′ proposed herein. Thus, in this extended system 10′, only the encoder portion of a transformer model known per se is used to generate a low-level representation 32 of the input data set. Instead of the transformer module, an autoencoder may also be provided, in which case again only its encoder portion is required and used. The first transformer module 30 thus generates a low-level representation 32 of the input data set from an input data set, wherein the first transformer module is trained such that the low-level representation 32 of the input data set represents the structure of the input data set 14 abstracted from the values contained in the input data set 14.

In order to assign values contained in the input data set 14 to the correct position in the desired data set in globally uniform, harmonized structure, the data sets 18 in globally uniform, harmonized structure are also transformed into different feature-reduced, abstracted representations 36 of the global eligible target structures using a second transformer model 34.

A transformer module that implements a transformer model for generating multiple low-level representations of a harmonized data set has the property that its encoder portion generates multiple low-level representations of the input data set of the transformer due to the self-attention layers. This property is used to perform pattern matching between a low-level representation 32 of the system's input data set 14 with different low-level representations 36 of a data set in the globally unified structure generated by the second transformer from the data set 18 in the globally unified structure as the second transformer's input data set.

Both the low-level representation 32 of a respective input data set 14 and the various feature-reduced, abstracted representations 36 of the eligible global target structures are fed to a pattern-matching module 38 configured to determine which of the feature-reduced, abstracted representations 36 of the eligible global target structure best matches the low-level representation 32 of the input data set 14. Since the feature-reduced, abstracted representations 36 of the eligible global target structures are derived from the data sets 18 in globally uniform, harmonized structure, the best mapping of the values from the input data set 14 to the appropriate target positions in the globally uniform, harmonized (target) structure can be performed using the most similar feature-reduced, abstracted representation 32 of the input data set 14 and the most similar feature-reduced, abstracted representations 36 of the eligible global target structures.

Each representation 36 of the global eligible target structures is a low-level representation formed by abstract feature vectors representing possible positions in the globally uniform harmonized (target) structure 18.

The abstract feature vectors (low-level representations) from the possible positions are compared to the low-level representation 32 of the input data sets by the pattern matching module 38 using a similarity metric. The similarity metric may be implemented as a distance measure, for example, or may be implemented as an approximated function by a neural network. The best position determined using the similarity metric is then selected as the target position for the corresponding values from the input data set 14. The result of the pattern matching is thus the positions of values from the input data set 14 in the corresponding data set 18 in a globally uniform, harmonized structure.

The target positions obtained for an input data set 14 using the pattern matching module 38 are then fed to the input layer of the harmonization module 16 along with the input data set 14. The harmonization module 16 then generates the desired data set 18 in a globally uniform, harmonized structure, which can then be further processed as described in connection with FIGS. 1 through 3.

In order to be able to use input data sets for different classifications or regressions, correspondingly different automated processing facilities 24.1, 24.2 and 24.3 can be provided; see FIG. 5. In this case, each automated processing facility 24.1, 24.2 and 24.3 is preferably preceded by its own preprocessing module 20.1, 20.2 and 20.3 in order to preprocess the data for the respective classification or regression model embodied by the automated processing facility in a model-specific manner.

The transfer into a uniform, harmonized data structure, on the other hand, can be performed centrally, which is why only one harmonization module 16 is required.

The models embodied by the harmonization module 16, the preprocessing module 20, and the automated processing facility 24 can typically be described by their structure or topology and by their parameterization. In the case of a neural network, the structure and topology of the respective neural network may be defined by a structure data set that includes, for example, information about how many layers the neural network has and what type of layers these layers are, how many nodes each layer has and how these are interconnected with nodes of adjacent layers, what activation function a respective node implements, etc. Such a structure dataset defines the neural network in both untrained and trained states.

By training the neural network, the weights are formed in the individual nodes, which determine how strongly output values of nodes of previous layers are taken into account by a node of a subsequent layer connected to them. The parameter values formed by training the neural network, i.e. in particular the weightings, can be stored in a parameter data set.

This makes it possible, for example, to transfer parameter values from a trained harmonization module 16 or preprocessing module 20 to another harmonization module 16 or preprocessing module 20 that has not been trained until then, provided that the respective embodied harmonization or preprocessing models have the same structure defined by a structure data set.

Accordingly, it is possible for both the harmonization models and the preprocessing models (each embodied by a harmonization module 16 or a preprocessing module 20) to be approximated in a decentralized manner across multiple instances using federated or collaborative learning. This is illustrated in FIGS. 6 and 7. Communication between individual preprocessing modules 20 or individual harmonization modules 16 can be done either directly from module to module or via a global server, which is shown as a cloud in FIGS. 6 and 7.

In an exemplary embodiment, the harmonization module has the structure of a four-layer perceptron with an input layer, two hidden layers and an output layer. Each of the layers has twelve nodes and the layers are fully connected. The activation function of the nodes is preferably a leaky ReLU function (ReLU: rectified linear unit). Accordingly, a structural data set associated with the harmonization module 16 describes such a four-layer perceptron. For example, if the four-layer perceptron is trained using reinforcement learning, the harmonization module 16 may also embody a deep Q-network (DQN).

The respective preprocessing module 20 preferably embodies an autoencoder for principal component analysis. The autoencoder has an input layer and an output layer and hidden layers in between, for example three hidden layers. The hidden layers have fewer nodes than the input and output layers. In a manner known per se, such an autoencoder is designed to optimize the weights in the nodes of the individual layers—for example by backpropagation—in such a way that, for example, a pixel matrix applied to the input layer is reproduced as similarly as possible by the output layer. This means that the deviation of the values of the corresponding nodes of the input layer and the output layer is minimized.

The weights formed at the nodes of a middle (hidden) layer during training represent the principal basis components of the input matrix. The middle layer has fewer nodes than either the input or the output layer. Input layer and output layer each have the same number of nodes.

The following application example illustrates how the system works: Six different clinics each provide input data sets.

For example, a particular input data set may contain medical history data for a patient (admission diagnosis, previous illnesses, age, place of residence, BMI, allergies, etc.) and various laboratory values (number of leukocytes, various antibody concentrations, etc.). In some cases, ECGs and medical images are also available for patients.

The task of the automated processing facilities is, for example, to determine the risk of infection with hospital germs on the basis of the input data sets, to determine the expected length of stay and to determine an expected value (score) for the expected risk of hospital germs. For each of these tasks, a separate automated processing facility 24.1, 24.2 and 24.3 may be provided (see FIG. 4), each embodying a decision model namely, for example, a classifier or regressor. Each of the decision models can be implemented as a parametric model (neural networks, logical regression, etc.) or as a non-parametric model (decision tree, support vector machines, gradient boosting trees, etc.). Model changes are implemented using prediction errors, preferably as a supervised learning algorithm.

In practice, it is often a problem that in a clinic A and F a different method is used for determining the leukocyte count than in the other clinics, which does not provide comparable values. Accordingly, these are also stored at a different position in the data model serving as the input data set. Likewise, all six data sets are stored in other information systems and database structures. Thus, all six data sets are available in a different standard.

Thus, the first task is to convert the input data sets into a harmonized data set format. This is done with the help of the harmonization module 16 and the harmonization model embodied by it (which can be, for example, a perceptron trained by reinforcement learning, see above).

During training, the harmonization model is updated based on the prediction errors of the three automated processing facilities 24.1, 24.2 and 24.3. The harmonization model 16, which is realized as a deep Q-network (DQN), is preferably updated by reinforcement learning via a reward based on the error values of the decision models embodied by the automated processing facilities 24.1, 24.2 and 24.3. For this purpose, a tree search is initially used to classify the different data formats and data standards into a global standard. The reward increases if the mapping consistently leads to an improvement of the harmonization model in all clinics.

For the leukocyte count, the harmonization model 16 is trained to split the values between two code systems. Equivalent treatment of the values from the different measurement methods results in a worse reward. The alternating decision models ensure that there is no overfitting in favor of one model. The DQN models are trained in a federated learning setup (see FIG. 7), which reduces clinic bias. The exchange between clinics allows to use already trained parameters and thus to achieve a transfer effect.

The respective pre-processing module 20.1, 20.2 or 20.3 provides for a selection of the relevant parameters and translates both types of leukocyte values into a uniform format. In particular, the relevant parameters are specific to the respective automated processing facility and the decision model embodied by it. The pre-processing model embodied by the pre-processing module can be implemented as an autoencoder, which is also federally trained, see FIG. 6.

### REFERENCE SIGN

10 System for automated harmonization of structured data from different collection facilities
12 Input of the system
14 Input data record in a recording device-specific structure
16 Harmonization module
18 Harmonized data set in a predefined, globally uniform, structure harmonized data structure
20 Preprocessing module
22 Data set with preprocessed data
24 Automated processing facility
26 Model specific data structure
30 Transformer module for generating a low-level representation of a respective input data set
32 Low-level representation of a respective input data set.
34 Transformer module for generating multiple low-level representations of a harmonized data structure
36 Low-level representation of a harmonized data structure
38 Pattern matching module

What is claimed is:

1. A system for automated harmonization of structured data from different acquisition devices, comprising:
an input for an input data set with heterogeneous data in an acquisition device-specific structure,
a harmonization module that embodies a harmonization model that is machine-generated and configured to convert a respective input data set in its respective acquisition-device-specific data structure into a harmonized data set in a predetermined, globally uniform data structure of the system,
a preprocessing module embodying a preprocessing model which is machine-generated and configured to convert data from a harmonized data set in the globally uniform data structure into preprocessed data in a model-specific data structure, in particular to perform feature reduction, and
an automated processing facility configured to automatically process data sets with preprocessed data in the model-specific data structure, in particular to classify and to generate a loss measure representing a possible processing inaccuracy (loss) for training the harmonization module and/or the preprocessing module and to selectively output the loss measure to the harmonization module or the preprocessing module.

2. The system according to claim 1, wherein the harmonization module embodies a trained neural network, in particular a multilayer fully-networked perceptron or a deep Q-network.

3. The system according to claim 1, wherein the preprocessing module embodies a trained neural network, in particular an autoencoder.

4. A system according to claim 1, wherein the harmonization module is connected to a plurality of preprocessing modules and each of the preprocessing modules is connected to an automated processing facility.

5. A system according to claim 1, wherein the automated processing facility for providing feedback to the harmonization module is connected to the harmonization module at least intermittently.

6. A system according to claim 1, wherein the automated processing facility is connected at least intermittently to the upstream preprocessing module for providing feedback thereto.

7. The system according to claim 1, wherein the preprocessing module is configured to convert data from a partial data set of a harmonized data set into a partial data set in which the data is present in a feature-reduced form.

8. The system according to claim 1, further comprising a transformer module for generating a low-level representation of a respective input data set.

9. The system according to claim 8, further comprising:
a second transformer module for generating a plurality of feature-reduced abstracted representations of a harmonized global target data structure; and
a pattern matching module configured to determine which of the feature-reduced abstracted representations of the global target structure in question best matches the low-level representation of the input data set.

10. A network of a plurality of systems according to claim 1 interconnected for exchanging parameter data sets containing parameter values representing weights generated by training of the harmonization or preprocessing models embodied by the harmonization or preprocessing modules to enable federated or collaborative machine learning.
